(19) United States
(12) Patent Application Publication
(10) Pub. No.: US 2024/0331834 A1
(43) Pub. Date: Oct. 3, 2024

(54) RADIATION TREATMENT PLANNING IMAGE PROVISIONING METHOD AND APPARATUS

(71) Applicant: Siemens Healthineers International AG, Steinhausen (CH)

(72) Inventors: Ari Harju, Espoo (FI); Petri Hiltunen, Espoo (FI)

(21) Appl. No.: 18/192,879

(22) Filed: Mar. 30, 2023

(51) Int. Cl. G16H 20/40; G06T 5/00; G06T 7/00; G06T 11/00

(52) U.S. Cl. CPC G16H 20/40; G06T 5/94; G06T 7/0012; G06T 11/003; G06T 2207/10081; G06T 2211/408

(57) ABSTRACT

A control circuit receives a radiation treatment planning system request for image data to support a particular radiation treatment planning step (typically for a particular corresponding patient). The control circuit then accesses particular image data that is particularly suitable to support the particular radiation treatment planning step and transmits that particular image data in response to the radiation treatment planning system request. Illustrative examples of particular radiation treatment planning steps include, but are not limited to, a contouring step, a segmenting step, a dose prediction step, and a dose calculation step, to note but a few. By one approach, and as one illustrative example, the aforementioned particular image data may comprise patient image information that includes segmented structures.

### TECHNICAL FIELD

These teachings relate generally to treating a patient's planning target volume with energy pursuant to an energy-based treatment plan and more particularly to optimizing an energy-based treatment plan as a function, at least in part, of patient image data.

### BACKGROUND

The use of energy to treat medical conditions comprises a known area of prior art endeavor. For example, radiation therapy comprises an important component of many treatment plans for reducing or eliminating unwanted tumors. Unfortunately, applied energy does not inherently discriminate between unwanted material and adjacent tissues, organs, or the like that are desired or even critical to continued survival of the patient. As a result, energy such as radiation is ordinarily applied in a carefully administered manner to at least attempt to restrict the energy to a given target volume. A so-called radiation treatment plan often serves in the foregoing regards.

A radiation treatment plan typically comprises specified values for each of a variety of treatment-platform parameters during each of a plurality of sequential fields. Treatment plans for radiation treatment sessions are often automatically generated through a so-called optimization process. As used herein, “optimization” will be understood to refer to improving a candidate treatment plan without necessarily ensuring that the optimized result is, in fact, the singular best solution. Such optimization often includes automatically adjusting one or more physical treatment parameters (often while observing one or more corresponding limits in these regards) and mathematically calculating a likely corresponding treatment result (such as a level of dosing) to identify a given set of treatment parameters that represent a good compromise between the desired therapeutic result and avoidance of undesired collateral effects.

Patient images are often employed when planning a radiation treatment plan. In particular, images (such as computed tomography and/or tomographic images) that depict treatment volumes, organs at risk, and other patient structures and features. Complications, inaccuracies, inefficiencies, and/or delays can occur when the supplied images are not inherently suitable to support a given corresponding radiation treatment planning step.

### BRIEF DESCRIPTION OF THE DRAWINGS

The above needs are at least partially met through provision of the radiation treatment planning image provisioning method and apparatus described in the following detailed description, particularly when studied in conjunction with the drawings, wherein:

FIG. 1 comprises a block diagram as configured in accordance with various embodiments of these teachings;

FIG. 2 comprises a block diagram as configured in accordance with various embodiments of these teachings;

FIG. 3 comprises a flow diagram as configured in accordance with various embodiments of these teachings; and

FIG. 4 comprises a flow diagram as configured in accordance with various embodiments of these teachings.

Elements in the figures are illustrated for simplicity and clarity and have not necessarily been drawn to scale. For example, the dimensions and/or relative positioning of some of the elements in the figures may be exaggerated relative to other elements to help to improve understanding of various embodiments of the present teachings. Also, common but well-understood elements that are useful or necessary in a commercially feasible embodiment are often not depicted in order to facilitate a less obstructed view of these various embodiments of the present teachings. Certain actions and/or steps may be described or depicted in a particular order of occurrence while those skilled in the art will understand that such specificity with respect to sequence is not actually required. The terms and expressions used herein have the ordinary technical meaning as is accorded to such terms and expressions by persons skilled in the technical field as set forth above except where different specific meanings have otherwise been set forth herein. The word “or” when used herein shall be interpreted as having a disjunctive construction rather than a conjunctive construction unless otherwise specifically indicated.

### DETAILED DESCRIPTION

Generally speaking, pursuant to these various embodiments a control circuit receives a radiation treatment planning system request for image data to support a particular radiation treatment planning step (typically for a particular corresponding patient). The control circuit then accesses particular image data that is particularly suitable to support the particular radiation treatment planning step and transmits that particular image data in response to the radiation treatment planning system request. Illustrative examples of particular radiation treatment planning steps include, but are not limited to, a contouring step, a segmenting step, a dose prediction step, and a dose calculation step, to note but a few. By one approach, and as one illustrative example, the aforementioned particular image data may comprise patient image information that includes segmented structures.

These teachings are flexible in practice and will accommodate various approaches as regards accessing the aforementioned particular image data that is particularly suitable to support the particular radiation treatment planning step. By one approach, the foregoing comprises accessing patient image information (such as, but not limited to, computed tomography image information and/or cone beam computed tomography image information) and then processing that patient image information to generate the aforementioned particular image data that is particularly suitable to support the particular radiation treatment planning step. As one simple example, the foregoing processing can comprise, at least in part, modifying image formatting.

As one illustrative example, when the particular radiation treatment planning step comprises a contouring step, the aforementioned processing can comprise, at least in part, enhancing contrast in the patient image information.

As another illustrative example, when the particular radiation treatment planning step comprises a dose calculation step, the aforementioned processing can comprise, at least in part, providing at least one of electron density information per patient voxel, mass density per patient voxel, relative proton stopping power per patient voxel, and/or volume material per patient voxel.

By one approach, the aforementioned processing can comprise, at least in part, reconstructing the particular image data from single energy computed tomography image data. By another approach, the aforementioned processing can comprise, at least in part, effective electron density reconstruction from dual energy computed tomography image data.

So configured, a radiation treatment planning system (automated, partially automated, or non-automated) can be timely provided with differentiated patient images that are each especially suitable to support the needs of a given radiation treatment planning step. This, in turn, can help to facilitate more rapid competition of the planning process and/or a more accurate therapeutic and efficacious result.

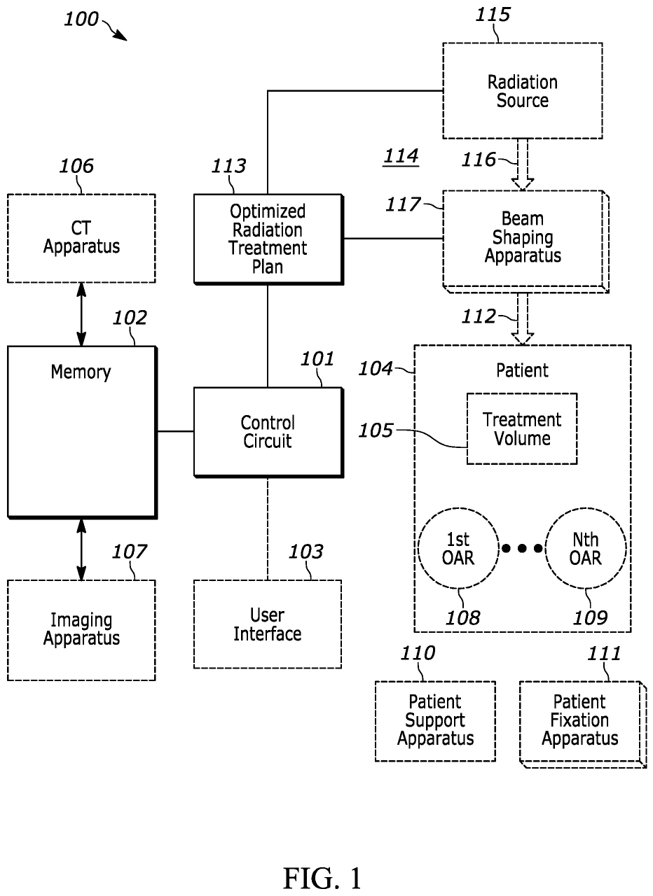

These and other benefits may become clearer upon making a thorough review and study of the following detailed description. Referring now to the drawings, and in particular to FIG. 1, an illustrative apparatus 100 and application setting that is compatible with many of these teachings will first be presented.

In this particular example, the apparatus 100 includes a control circuit 101. Being a “circuit,” the control circuit 101 therefore comprises structure that includes at least one (and typically many) electrically-conductive paths (such as paths comprised of a conductive metal such as copper or silver) that convey electricity in an ordered manner, which path(s) will also typically include corresponding electrical components (both passive (such as resistors and capacitors) and active (such as any of a variety of semiconductor-based devices) as appropriate) to permit the circuit to effect the control aspect of these teachings.

Such a control circuit 101 can comprise a fixed-purpose hard-wired hardware platform (including but not limited to an application-specific integrated circuit (ASIC) (which is an integrated circuit that is customized by design for a particular use, rather than intended for general-purpose use), a field-programmable gate array (FPGA), and the like) or can comprise a partially or wholly-programmable hardware platform (including but not limited to microcontrollers, microprocessors, and the like). These architectural options for such structures are well known and understood in the art and require no further description here. This control circuit 101 is configured (for example, by using corresponding programming as will be well understood by those skilled in the art) to carry out one or more of the steps, actions, and/or functions described herein.

The control circuit 101 operably couples to a memory 102. This memory 102 may be integral to the control circuit 101 or can be physically discrete (in whole or in part) from the control circuit 101 as desired. This memory 102 can also be local with respect to the control circuit 101 (where, for example, both share a common circuit board, chassis, power supply, and/or housing) or can be partially or wholly remote with respect to the control circuit 101 (where, for example, the memory 102 is physically located in another facility, metropolitan area, or even country as compared to the control circuit 101).

In addition to information such as patient image information, other optimization information for a particular patient, and information regarding a particular radiation treatment platform as described herein, this memory 102 can serve, for example, to non-transitorily store the computer instructions that, when executed by the control circuit 101, cause the control circuit 101 to behave as described herein. (As used herein, this reference to “non-transitorily” will be understood to refer to a non-ephemeral state for the stored contents (and hence excludes when the stored contents merely constitute signals or waves) rather than volatility of the storage media itself and hence includes both non-volatile memory (such as read-only memory (ROM) as well as volatile memory (such as a dynamic random access memory (DRAM).)

By one optional approach the control circuit 101 also operably couples to a user interface 103. This user interface 103 can comprise any of a variety of user-input mechanisms (such as, but not limited to, keyboards and keypads, cursor-control devices, touch-sensitive displays, speech-recognition interfaces, gesture-recognition interfaces, and so forth) and/or user-output mechanisms (such as, but not limited to, visual displays, audio transducers, printers, and so forth) to facilitate receiving information and/or instructions from a user and/or providing information to a user.

If desired the control circuit 101 can also operably couple to a network interface (not shown). So configured the control circuit 101 can communicate with other elements (both within the apparatus 100 and external thereto) via the network interface. Network interfaces, including both wireless and non-wireless platforms, are well understood in the art and require no particular elaboration here.

By one approach, a computed tomography apparatus 106 and/or other imaging apparatus 107 as are known in the art can source some or all of any desired patient-related imaging information.

In this illustrative example the control circuit 101 is configured to ultimately output an optimized energy-based treatment plan (such as, for example, an optimized radiation treatment plan 113). This energy-based treatment plan typically comprises specified values for each of a variety of treatment-platform parameters during each of a plurality of sequential exposure fields. In this case the energy-based treatment plan is generated through an optimization process, examples of which are provided further herein.

By one approach the control circuit 101 can operably couple to an energy-based treatment platform 114 that is configured to deliver therapeutic energy 112 to a corresponding patient 104 in accordance with the optimized energy-based treatment plan 113. The patient 104 may have a treatment volume 105 and one or more organs-at-risk (OAR) as represented by a 1st OAR through an Nth OAR (denoted by the reference numerals 108 and 109). These teachings are generally applicable for use with any of a wide variety of energy-based treatment platforms/apparatuses. In a typical application setting the energy-based treatment platform 114 will include an energy source such as a radiation source 115 of ionizing radiation 116.

By one approach this radiation source 115 can be selectively moved via a gantry along an arcuate pathway (where the pathway encompasses, at least to some extent, the patient themselves during administration of the treatment). The arcuate pathway may comprise a complete or nearly complete circle as desired. By one approach the control circuit 101 controls the movement of the radiation source 115 along that arcuate pathway, and may accordingly control when the radiation source 115 starts moving, stops moving, accelerates, de-accelerates, and/or a velocity at which the radiation source 115 travels along the arcuate pathway.

As one illustrative example, the radiation source 115 can comprise, for example, a radio-frequency (RF) linear particle accelerator-based (linac-based) x-ray source. A linac is a type of particle accelerator that greatly increases the kinetic energy of charged subatomic particles or ions by subjecting the charged particles to a series of oscillating electric potentials along a linear beamline, which can be used to generate ionizing radiation (e.g., X-rays) 116 and high energy electrons.

A typical energy-based treatment platform 114 may also include one or more support apparatuses 110 (such as a couch) to support the patient 104 during the treatment session, one or more patient fixation apparatuses 111, a gantry or other movable mechanism to permit selective movement of the radiation source 115, and one or more energy-shaping apparatuses (for example, beam-shaping apparatuses 117 such as jaws, multi-leaf collimators, and so forth) to provide selective energy shaping and/or energy modulation as desired.

In a typical application setting, it is presumed herein that the patient support apparatus 110 is selectively controllable to move in any direction (i.e., any X, Y, or Z direction) during an energy-based treatment session by the control circuit 101. As the foregoing elements and systems are well understood in the art, further elaboration in these regards is not provided here except where otherwise relevant to the description.

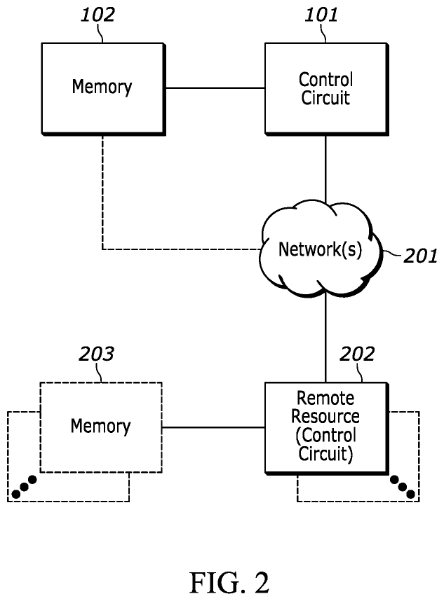

By one approach, and referring now to FIG. 2, the apparatus 100 may further provide for communicatively coupling the aforementioned control circuit 101 (and/or the aforementioned memory 102) via one or more intervening networks 201 to one or more remote resources 202. This remote resource 202 may itself comprise a control circuit as is essentially described above. This remote resource 202 may be physically remote from the facility that houses the aforementioned control circuit 101, or may physically share the same facility as desired. This remote resource 202 may also communicate with the aforementioned memory 102 and/or with other memory 203 that is physically apart from the apparatus 100. These memories 102, 203 may contain, for example, patient image information and/or particular image data that is particularly suitable to support corresponding particular radiation treatment planning steps as discussed herein.

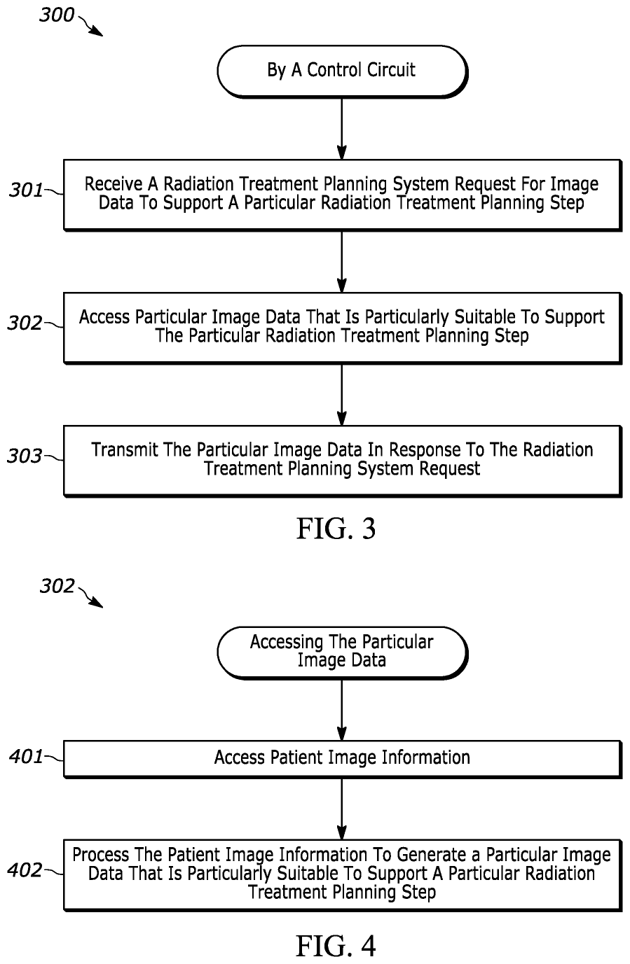

Referring now to FIG. 3, a process 300 that can be carried out, for example, in conjunction with the above-described application setting (and more particularly via one or both of the aforementioned control circuits 101, 202, hereinafter simply referred to as “a control circuit” or “the control circuit”) will be described. Generally speaking, this process 300 serves to facilitate generating an optimized radiation treatment plan 113 to thereby facilitate treating a particular patient with therapeutic radiation using a particular radiation treatment platform per that optimized radiation treatment plan.

At block 301, the control circuit receives a radiation treatment planning system request (i.e., a request from a radiation treatment planning system) for image data to support a particular radiation treatment planning step. This request may be sourced external to the control circuit or may be an internal request from, for example, radiation treatment planning system automated software being run by the control circuit. These teachings are intended to encompass a variety of different radiation treatment planning steps. Some nonlimiting examples in these regards include a contouring step, a segmenting step, a dose prediction step, and a dose calculation step.

In response to receiving the foregoing request, the control circuit, at block 302, accesses particular image data that is particularly suitable to support the aforementioned particular radiation treatment planning step. As used herein, this reference to image data being “particularly suitable” to support a particular radiation treatment planning step shall be understood to refer to the image data being in a form that is more helpful to the planning step than the image data being in a different (perhaps original) form. To be “helpful,” the image has a form that allows the planning step to be carried out with greater accuracy, timeliness, and/or compatibility with the processing details of the planning step itself.

As one illustrative example, this particular image data might comprise patient imagery that includes segmented structures (such as identified targeted volumes, organs at risk, and/or other tissues and structures in the patient's body).

These teachings are flexible in practice and will accommodate various approaches in the foregoing regards. Referring momentarily to FIG. 4, details regarding some of these approaches will be presented. It will be understood that these details are intended to serve an illustrative purpose and are not intended to suggest any limitations with respect to these teachings.

At block 401, the control circuit accesses patient image information. Those skilled in the art will appreciate that this activity may occur prior to receiving the above-described radiation treatment planning system request or may occur in response to receiving that request. This patient image information may comprise, by way of example, computed tomography image information and/or cone beam computed tomography image information (as initially sourced, for example, by the aforementioned CT apparatus 106 and/or imaging apparatus 107).

At block 402, the control circuit processes that patient image information to generate particular image data that is particularly suitable to support a corresponding particular radiation treatment planning step (such as the aforementioned particular radiation treatment planning step that corresponds to the radiation treatment planning system request). And again, those skilled in the art will appreciate that this processing activity may occur prior to receiving the above-described radiation treatment planning system request or may occur in response to receiving that request, as desired. These teachings will also accommodate at least partially processing patient image information prior to a time of need, and then, at a time of need, further processing that partially processed patient information in order to provide particular image data to support a corresponding particular radiation treatment planning step.

As one illustrative example, the foregoing processing may comprise, at least in part, modifying image formatting. By one approach, the foregoing processing may comprise, at least in part, reconstructing from single energy computed tomography image data. By another approach, when the patient image information comprises dual energy computed tomography image data, the foregoing processing may comprise generating any one or more of:
effective electron density images (including electron density maps);
effective atomic number images (including effective atomic number (Zeff) maps;
weighted average images (simulating single energy spectra);
virtual monoenergetic images (attenuation at a single photon energy rather than a spectrum);
material decomposition images (mapping or removing substances of known attenuation characteristics, such as iodine, calcium, or uric acid);
virtual non-contrast images (for example, iodine removed);
iodine concentration images (for example, iodine maps);
calcium suppression images (calcium removed); and
uric acid suppression images (uric acid removed).

As a more specific illustrative example, when the particular radiation treatment planning step comprises a contouring step, the foregoing processing may comprise, at least in part, enhancing contrast in the patient image information.

As another more specific illustrative example, when the particular radiation treatment planning step comprises a dose calculation step, the foregoing processing may comprise, at least in part, generating particular image data that provides at least one of electron density information per patient voxel, mass density per patient voxel, relative proton stopping power per patient voxel, and volume material per patient voxel (wherein the volume material serves to identify the material makeup of the corresponding volume).

Referring again to FIG. 3, at block 303, the control circuit transmits the particular image data in response to the radiation treatment planning system request. It may be noted that when the aforementioned processing occurs prior to initiation of the radiation treatment planning process, the overall process may be carried out more quickly because no time need be spent on preparing such images at the time of need.

The radiation treatment planning system that sourced the foregoing request can then employ the received particular image data to carry out the particular radiation treatment planning step that corresponds to that information. The radiation treatment planning system can make subsequent additional requests for additional image data to support other radiation treatment planning steps, and the foregoing activities can be repeated in order to generate/provide the appropriate image data to support the corresponding step.

Upon concluding the radiation treatment planning process, the resultant optimized radiation treatment plan 113 can be utilized with the aforementioned radiation treatment platform 114 to administer therapeutic energy 112 to one or more treatment volumes 105 in the patient 104.

So configured, these teachings can support providing a centralized image service that generates/stores/delivers patient images particularly suitable to support a variety of use cases during radiation treatment planning. These teachings can greatly streamline and/or integrate imaging and radiation treatment planning workflows and can help to avoid application settings where the radiation treatment planning system simply imports one patient image with which the system must then make do.

Those skilled in the art will recognize that a wide variety of modifications, alterations, and combinations can be made with respect to the above described embodiments without departing from the scope of the invention. For example, if desired, these teachings could be employed in application settings other than radiation treatment planning. Accordingly, such modifications, alterations, and combinations are to be viewed as being within the ambit of the inventive concept.

What is claimed is:

1. An apparatus comprising:
a control circuit configured to:
receive a radiation treatment planning system request for image data to support a particular radiation treatment planning step;
access particular image data that is particularly suitable to support the particular radiation treatment planning step;
transmit the particular image data in response to the radiation treatment planning system request.

2. The apparatus of claim 1, wherein the particular radiation treatment planning step comprises one of:
a contouring step;
a segmenting step;
a dose prediction step;
a dose calculation step.

3. The apparatus of claim 1, wherein the particular image data comprises patient image information that includes segmented structures.

4. The apparatus of claim 1, wherein the control circuit is configured to access the particular image data that is particularly suitable to support the particular radiation treatment planning step by:
accessing patient image information;
processing the patient image information to generate the particular image data that is particularly suitable to support the particular radiation treatment planning step.

5. The apparatus of claim 4, wherein the patient image information comprises at least one of computed tomography image information and cone beam computed tomography image information.

6. The apparatus of claim 4, wherein the control circuit is configured to process the patient image information to generate the particular image data by, at least in part, modifying image formatting.

7. The apparatus of claim 4, wherein the particular radiation treatment planning step comprises a contouring step and wherein the control circuit is configured to process the patient image information to generate the particular image data by, at least in part, enhancing contrast in the patient image information.

8. The apparatus of claim 4, wherein the particular radiation treatment planning step comprises a dose calculation step and wherein the control circuit is configured to process the patient image information to generate the particular image data by, at least in part, providing at least one of:
electron density information per patient voxel;
mass density per patient voxel;
relative proton stopping power per patient voxel;
volume material per patient voxel.

9. The apparatus of claim 4, wherein the control circuit is configured to process the patient image information to generate the particular image data that is particularly suitable to support the particular radiation treatment planning step by, at least in part, reconstructing the particular image data from single energy computed tomography image data.

10. The apparatus of claim 4, wherein the control circuit is configured to process the patient image information to generate the particular image data that is particularly suitable to support the particular radiation treatment planning step by, at least in part, effective electron density reconstruction from dual energy computed tomography image data.

11. A method comprising:
by a control circuit:
receiving a radiation treatment planning system request for image data to support a particular radiation treatment planning step;
accessing particular image data that is particularly suitable to support the particular radiation treatment planning step;
transmitting the particular image data in response to the radiation treatment planning system request.

12. The method of claim 11, wherein the particular radiation treatment planning step comprises one of:
a contouring step;
a segmenting step;
a dose prediction step;
a dose calculation step.

13. The method of claim 11, wherein the particular image data comprises patient image information that includes segmented structures.

14. The method of claim 11, wherein accessing the particular image data that is particularly suitable to support the particular radiation treatment planning step comprises:
accessing patient image information;
processing the patient image information to generate the particular image data that is particularly suitable to support the particular radiation treatment planning step.

15. The method of claim 14, wherein the patient image information comprises at least one of computed tomography image information and cone beam computed tomography image information.

16. The method of claim 14, wherein processing the patient image information to generate the particular image data comprises, at least in part, modifying image formatting.

17. The method of claim 14, wherein the particular radiation treatment planning step comprises a contouring step and wherein processing the patient image information to generate the particular image data comprises, at least in part, enhancing contrast in the patient image information.

18. The method of claim 14, wherein the particular radiation treatment planning step comprises a dose calculation step and wherein processing the patient image information to generate the particular image data comprises, at least in part, providing at least one of:
electron density information per patient voxel;
mass density per patient voxel;
relative proton stopping power per patient voxel;
volume material per patient voxel.

19. The method of claim 14, wherein processing the patient image information to generate the particular image data that is particularly suitable to support the particular radiation treatment planning step comprises, at least in part, reconstructing the particular image data from single energy computed tomography image data.

20. The method of claim 14, wherein processing the patient image information to generate the particular image data that is particularly suitable to support the particular radiation treatment planning step comprises, at least in part, effective electron density reconstruction from dual energy computed tomography image data.
